(19) United States
(12) Patent Application Publication
(10) Pub. No.: US 2005/0241011 A1
(43) Pub. Date: Oct. 27, 2005

(54) Enclosed aquacultural systems for production of purified recombinant proteins

(76) Inventors: F C Thomas Allnut, Glenwood, MD (US); Moti Harel, Baltimore, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/510,624

(22) PCT Filed: Apr. 8, 2003
(86) PCT No.: PCT/US03/10772

§ 371 Date: May 6, 2005

Related U.S. Application Data
(60) Provisional application No. 60/370,689, filed on Apr. 9, 2002.

(51) Int. Cl. A01K 67/033; A01K 67/027

(52) U.S. Cl. 800/13; 800/20

(57) ABSTRACT

The invention provides a production method for purified recombinant proteins of commercial value prepared in enclosed aquatic systems. The production system provides biosecure containment of genetically engineered aquatic organisms for an alternative production method for complex proteins requiring post-translational modification that is best done in eukaryotic expression systems. Possible aquatic organisms that can be utilized by this invention are crustaceans (e.g. penaeid shrimp or Artemia) and teleost fish (e.g. trout or tilapia).

### BACKGROUND OF THE INVENTION

This invention relates to recombinant proteins produced in aquatic organisms that are grown in enclosed aquacultural systems. This invention also relates to a method for producing a recombinant protein in an aquatic organism grown in an enclosed aquacultural system.

The importance of recombinant proteins for modern medical applications and therapy cannot be overemphasized. Recombinant production methods for bacteria are well developed (Jonasson, Liljeqvist et al. 2002). Many important commercial proteins are produced in bacterial prokaryotic systems, having importance in industry and medical science. As the critical nature of post-translational modification has become apparent (Ho, Gilbert et al. 1999), eukaryotic expression systems are being evaluated for their ability to allow production of recombinant proteins that undergo post-translational modification in a manner analogous to the natural production system (e.g. human or mammalian systems). The initial evaluation of yeasts for this purpose (Cereghino and Cregg 1999; Cereghino and Cregg 2000; Cregg, Cereghino et al. 2000), while an improvement over bacterial expression systems, has not provided the required post-translational modification for maximal activity. Mammalian cell culture has been the most successful in replication of the proper post-translational modification of recombinantly expressed mammalian proteins. However, mammalian cell culture is expensive and not easily scaled up for commercial production of therapeutic proteins. For this reason, alternative production systems are being sought. The use of insect cell culture, based on stable transformation or viral transfection methods, has been a recent approach that is partially successful (Miller 1988; Cha, Dalal et al. 1999; Cha, Dalal et al. 1999; Platteborze and Broomfield 2000). The post-translational modification in insect cell and larval culture has not yet achieved the required standard set by mammalian expression. Methods are being developed in insect cell culture systems to provide N-terminal sialyation for expression of proteins more like the mammalian-expressed protein. However, the expression in insect cell culture and larval culture has still not provided the methods for properly processed proteins in an economical process.

Aquaculture as an industry is rapidly being developed for production of biomass for food (e.g., shrimp and fish farming) (Halvorson and Quezada 1999). As these methods begin to impact the environment, biosecure and recirculating systems are also under development (Pruder, Moss et al. 2001). This is leading to completely isolated, inland systems that protect both the food being produced and the external environment from wastes and non-traditional methods of food production (e.g., unintentional release of genetically modified organisms).

The methods for recombinantly manipulating aquatic organisms are still in their infancy. Methods for transformation of a number of aquatic organisms are already available while recombinant proteins have been expressed in many non-aquatic systems. Mainly growth hormones and insulin have been expressed in fish. In one system, the islet cells of tilapia were humanized then harvested from the fish for xenotransplantation into model organisms (Wright and Pohajdak 2000). However, contained aquatic systems for the production of purified recombinant proteins for commercial or therapeutic applications have not been used prior to the instant invention.

Thus, there is a need for new methods for expression of recombinant proteins that undergo post-translational modification in a manner analogous to the mammalian or source organism, that are properly contained for biosecurity, and that reduce the overall cost of the purified recombinant protein.

### SUMMARY OF THE INVENTION

This invention aids in fulfilling these needs in the art. In one aspect, the invention provides methods for producing purified recombinant proteins utilizing aquatic systems in a biosecure recirculating system, thereby overcoming the problem of mass production.

In another aspect, the invention provides isolated recombinant proteins from biomass grown in these aquatic systems. The proteins can be isolated to partial or complete homogeneity.

These and other aspects of the invention are provided by one or more of the following embodiments.

In one aspect, the invention provides a recombinant aquatic organism cultured in an enclosed aquatic system, which produces an exogenous protein.

In one embodiment, the aquatic organism can be a commercially cultured fish, e.g., tilapia, sea bream, bass, zebrafish, rainbow trout, carp, cod, catfish, salmon, yellow tail, or red drum; a crustacean, e.g. a shrimp, including the brine shrimp Artemia; or a rotifer.

In another embodiment, the recombinant protein of the aquatic organism is post-translationally modified. The post-translational modification can be added by the cellular machinery of the host cell or by other heterologous proteins that are co-expressed with the recombinant protein.

In yet another embodiment, the recombinant aquatic organism produces a protein. Any protein can be produced using the present invention. Nonlimiting examples include thrombin, e.g., human thrombin; fibrinogen; green fluorescent protein; and acidic ribosomal phosphoprotein or fusions thereof.

In a further embodiment, the enclosed aquatic system is either completely isolated or is partially isolated from the external environment.

In another aspect, the invention provides a method for producing an isolated recombinant protein in an aquatic organism, in which DNA encoding an exogenous protein is introduced into the aquatic organism, the organism is cultured, harvested, and the recombinant protein isolated.

In another embodiment, the aquatic organism is transformed with a baculovirus vector, e.g. Autographa californica nuclear polyhedrosis virus. This embodiment includes all forms of the virus, plasmids, cosmids, phagemids, phages, naked DNA, and naked RNA.

In yet another embodiment, the method provides DNA with a promoter that is functional in the aquatic organism. The promoter can be any suitable promoter, such as an early/intermediate promoter, or a late promoter.

In a further embodiment, the method produces a protein with a post-translational modification. Likewise, the method can produce a protein without a post-translational modification.

DNA can be introduced into the organism by any suitable method. Methods for introducing DNA include, but are not limited to, infection and microinjection.

In another embodiment, the DNA is introduced into an Artemia cyst by microinjection. The cyst can be decapsulated, hatched, and cultured to produce additional recombinant Artemia. The cysts can be harvested by centrifugation.

In yet another embodiment, the recombinant aquatic organism is cultured in an enclosed aquatic system that is either completely isolated or is partially isolated. This enclosed aquatic system can be recirculating.

In a further embodiment, the recombinant aquatic organism is harvested. Methods of harvesting include, but are not limited to, nets and filtration.

In yet a further embodiment, the recombinant aquatic organism is rapidly frozen upon harvest.

In another embodiment, the recombinant protein is thrombin, e.g., human thrombin; fibrinogen; green fluorescent protein; or acidic ribosomal phosphoprotein or fusions thereof.

In yet another embodiment, the recombinant protein is targeted to a specific tissue, for example, muscle or skin, by placing the exogenous DNA under the control of a regulatory promoter endogenous to that tissue.

In a further embodiment, a crustacean expressing a recombinant protein or peptide is used to prophylactically and therapeutically treat a human or non-human animal.

In yet a further embodiment, the isolated recombinant protein or peptide is used to prophylactically or therapeutically treat a human or non-human animal.

The inventors have discovered that recombinant proteins can be expressed in biosecure, aquatic systems in a method that provides the unexpected advantage of proper post-translational modification for optimal production of the protein, control of all external factors affecting the level of expression in the aquatic system, economics of scale over existing cell culture methods, and flexibility in both the host organism and the tissues targeted for expression of a wide variety of recombinant proteins.

### DETAILED DESCRIPTION OF THE EMBODIMENTS OF THE INVENTION

Definitions

An “aquatic organism,” as utilized in this invention, is an organism that is grown in water, either fresh- or saltwater, excluding prokaryotic organisms, plants, algae, and single-celled organisms or cell culture. Aquatic organisms, as contemplated under this invention, include, but are not limited to, the following organisms; fish, such as bass, striped bass, tilapia, catfish, sea bream, rainbow trout, zebrafish, red drum and carp; crustaceans, such as penaeid shrimp, brine shrimp, freshwater shrimp, and Artemia; and rotifers.

An “enclosed aquatic system” is a system wherein the input and outputs in nutrients, water, and other components are controlled either completely or partially to effect at least partial isolation of the production system from the outside environment. Complete and partial isolation of the system are envisioned in this invention. Such a production system differs from aquaria, ponds, and other non-commercial aquatic growth systems in being based on the intensive culture of marine organisms that are selected to be specifically pathogen free. The system is biosecure with facilities effectively disinfected and isolated from sources of disease vectors and environmental factors (and conversely the environment is protected from what is in the biosecure system), the microbial flora is manipulated to provide a beneficial, synergistic microbial population for growth of the marine organism, and the aqueous medium is carefully controlled in composition. Zero-exchange of the aqueous medium (or minimal exchange) is maintained to prevent contamination of the system as well as to obtain maximal production. Such a system for shrimp is detailed in U.S. Pat. No. 6,327,996 by Pruder et al (2001).

### EXAMPLES

Certain embodiments of the invention will now be described in more detail through the following examples. The examples are intended solely to aid in more fully describing selected embodiments of the invention, and should not be considered to limit the scope of the invention in any way.

### Example 1

### Production of Thrombin in Artemia Using Viral Transformation

Artemia nauplii are transformed using a viral transformation system based on commercially available Autographa californica nuclear polyhedrosis virus (AcMNPV, herein referred to as baculovirus systems) for transfection of alternative arthropod systems. The baculovirus vectors are engineered to express DNA containing the human form of the thrombin gene (Genback AF478696) on infection of the Artemia using standard molecular biological methods (Sambrook, Fritsch et al. 1989, for example). The time for harvest to obtain maximal production of the thrombin is determined empirically in a small-scale enclosed culture system and depends on the type of baculovirus promoter used to drive expression. The early/intermediate (gp64) or late (polh) promoters of the baculovirus system determine when maximal protein expression occurs. Late promoters allow more overall production of the recombinant protein but the early/intermediate promoters may provide better post-translational modification. Artemia are harvested by filtration and biomass rapidly frozen in dry ice or liquid nitrogen. Thrombin can be isolated from the frozen whole Artemia using standard methods (Lundblad, Kingdon et al. 1976). This purified material can be utilized for therapeutic applications, such as wound healing applications.

### Example 2

### Production of Fibrinogen in the Muscle of Striped Bass

The genes for fibrinogen (Chung, Harris et al. 1991) are cloned into a vector allowing multiple transcript expression in fish. The location of muscle regulatory genes has recently been documented (Tan, Hoang et al. 2002; Tan 2002), allowing development of molecular constructs with specific targeting of foreign protein expression in the muscle of fish. Using standard methods in molecular biology, the fibrinogen genes are placed in a molecular construct that allows expression of the genes for fibrinogen in striped bass, driven by endogenous muscle regulatory promoters. Methods used for the transformation of zebrafish embryos, such as microinjection into fertilized eggs, can be applied to this system (Nasevicius and Ekker 2001). Alternative methods such as viral transfection or microparticle bombardment could also be applied to affect transformation of the fish and/or fish embryos. The fish will be raised in enclosed, recirculating culture systems from the larval to at least the F1 generation prior to harvest for recombinant protein production. Larger bass will yield higher amounts of muscle per fish, but might not provide the highest ratio of expressed recombinant protein relative to total biomass. Fish are harvested with nets and either processed as raw biomass or processed to separate muscle tissue from the rest of the biomass. Biomass or muscle tissue can either be treated for production immediately or flash frozen for future processing. The biomass or muscle tissue is treated with proteinase inhibitors and fibrinogen isolated as described previously (Gaffney 1972). Purified fibrinogen can be then utilized as a therapeutic for wound healing and other medical applications.

### Example 3

### Production of Fibrinogen in the Skin of Rainbow Trout

The genes for fibrinogen (Chung, Harris et al. 1991) are cloned into a vector allowing multiple transcript expression and targeted to the skin of rainbow trout. Various promoters have been identified that target the protein to skin. In zebrafish, the type II cytokeratin (CK) gene promoter targets the gene to the juvenile skin epithelia or to adult skin (Ju, Xu et al. 1999). The homologous gene can be isolated from the rainbow trout and utilized for construction of an expression vector using standard techniques (Sambrook, Fritsch et al. 1989). The rainbow trout embryo is transformed by microinjection using this construct as described for zebrafish embryos (Nasevicius and Ekker 2001). Expression of the fibrinogen genes in the skin will occur and allow one to harvest the protein for purification as described in Example 2.

### Example 4

### Production of Green Fluorescent Protein in Zebrafish Skin

The type II cytokeratin (CK) gene from zebrafish is used to construct a vector containing the GFP gene under the control of the CK promoter. This is then used to transform the zebra fish embryo using standard methods (Nasevicius and Ekker 2001). GFP is then expressed in the skin of the fish and can be isolated directly via affinity chromatography using anti-GFP from commercial sources covalently bound to a cyanogen bromide activated SEPHADEX column (Amersham Pharmacia Biotech).

### Example 5

### Generalized Expression of Foreign Protein in Zebrafish

The acidic ribosomal phosphoprotein PO (arp) gene is expressed ubiquitously in this fish (Ju, Xu et al. 1999). Placing this gene in a vector using standard methods and insertion of a foreign protein in frame behind this promoter would allow production of the protein throughout the body of the fish. Proteins expressed in this manner could then be isolated by standard methods.

### Example 6

### Production of Thrombin in Artemia Using Microinjection of Decapsulated Cysts

Thrombin is produced in Artemia as in Example 1, except the Artemia cysts are transformed by microinjecting the constructs containing the thrombin genes using standard methods applied to fish embryos for microinjection (Nasevicius and Ekker 2001). The recombinant Artemia are reared in a recirculating system, forced to encyst, and then stored or used generate additional encysted recombinant organisms. The cysts are then utilized in a production method for the recombinant thrombin. Cysts are decapsulated, hatched, and raised in an enclosed aquatic system until very high density of biomass is achieved. They are then harvested by centrifugation, filtration, or alternative means. The recombinant Artemia are then processed to extract the recombinant protein using standard isolation methodology. Purified or partially purified recombinant proteins can then be used for therapeutic applications (Deutscher 1990).

### REFERENCES

Cereghino, G. P. and J. M. Cregg (1999). “Applications of yeast in biotechnology: protein production and genetic analysis.” Curr Opin Biotechnol 10 (5): 422-7.
Cereghino, J. L. and J. M. Cregg (2000). “Heterologous protein expression in the methylotrophic yeast Pichia pastoris.” FEMS Microbiol Rev 24 (1): 45-66.
Cha, H. J., N. G. Dalal, et al. (1999). “Insect larval expression process is optimized by generating fusions with green fluorescent protein.” Biotechnol Bioena 65 (3): 316-24.
Cha, H. J., N. G. Dalal, et al. (1999). “Expression and purification of human interleukin-2 simplified as a fusion with green fluorescent protein in suspended Sf-9 insect cells.” J. Biotechnol. 69 (1): 9-17.
Chung, D. W., J. E. Harris, et al. (1991). Nucleotide sequences of the three genes coding for human fibrinogen. Fibrinogen, Thrombosis, Coagulation and Fibrinolysis. C. Y. Liu and S. Chien: 39-48.
Cregg, J. M., J. L. Cereghino, et al. (2000). “Recombinant protein expression in Pichia pastoris.” Mol. Biotechnol. 16 (1): 23-52.
Deutscher, M. (1990). Guide to protein purification. New York, Academic Press.
Gaffney, P. J. (1972). “Localization of carbohydrate in the subunits of human fibrinogen and its plasmin induced fragments.” Biochim. Biophys. Acta 263 (2): 453-8.
Halvorson, H. and F. Quezada (1999). “Increasing public involvement in enriching our fish stocks through genetic enhancement.” Genet. Anal. 15 (3-5): 75-84.
Ho, H. H., M. T. Gilbert, et al. (1999). “Glycosylation is important for binding to human calcitonin receptors.” Biochemistry 38 (6): 1866-72.
Jonasson, P., S. Liljeqvist, et al. (2002). “Genetic design for facilitated production and recovery of recombinant proteins in Escherichia coli.” Biotechnol. Appl. Biochem. 35 (Pt 2): 91-105.
Ju, B., Y. Xu, et al. (1999). “Faithful expression of green fluorescent protein (GFP) in transgenic zebrafish embryos under control of zebrafish gene promoters.” Dev. Genet. 25 (2): 158-67.
Lundblad, R. L., H. S. Kingdon, et al. (1976). “Thrombin.” Methods Enzmmol. 45: 156-76.
Miller, L. K. (1988). “Baculoviruses as gene expression vectors.” Annu. Rev. Microbiol. 42: 177-99.
Nasevicius, A. and S. C. Ekker (2001). “The zebrafish as a novel system for functional genomics and therapeutic development applications.” Curr. Opin. Mol. Ther. 3 (3): 224-8.
Platteborze, P. L. and C. A. Broomfield (2000). “Expression of biologically active human butyrylcholinesterase in the cabbage looper (Trichoplusia ni).” Biotechnol. Appl. Biochem. 31 (Pt 3): 225-9.
Pruder, G. D., S. M. Moss, et al. (2001). Biosecure zero-exchange system for maturation and growout of marine animals. U.S. Pat. No. 6,327,996.
Sambrook, J., E. Fritsch, et al. (1989). Molecular Cloning: A laboratory manual. Cold Spring Harbor, Cold Spring Harbor Press.
Tan, X., L. Hoang, et al. (2002). “Characterization of muscle-regulatory genes, myf5 and myogenin, from striped bass and promoter analysis of muscle-specific expression.” Mar. Biotech. in press.
Tan, X. a. D., S. J. (2002). “Differential expression of two MyoD genes in fast and slow muscles of gilthead seabream (Sparus aurata).” Devel. Genes Evol. in press.
Wright, J. and B. Pohajdak (2000). Transgenic fish and a method of harvesting islet cells therefrom. U.S. Pat. No. 6,015,713, Dalhousie University.

1. An aquatic organism cultured in an enclosed aquatic system, comprising an exogenous recombinant protein.

2. The aquatic organism of claim 1, comprising a commercially cultured fish.

3. The aquatic organism of claim 2, wherein the fish is selected from tilapia, sea bream, bass, zebrafish, rainbow trout, carp, cod, catfish, salmon, yellow tail, and red drum.

4. The aquatic organism of claim 1, comprising a crustacean.

5. The aquatic organism of claim 4, wherein the crustacean is an Artemia.

6. The aquatic organism of claim 4, wherein the crustacean is a shrimp.

7. The aquatic organism of claim 1, comprising a rotifer.

8. The aquatic organism of claim 1, wherein the recombinant protein comprises a post-translational modification.

9. The aquatic organism of claim 1, wherein the recombinant protein is thrombin.

10. The aquatic organism of claim 9, wherein the recombinant protein is human thrombin.

11. The aquatic organism of claim 1, wherein the recombinant protein is fibrinogen.

12. The aquatic organism of claim 1, wherein the recombinant protein is green fluorescent protein or a fusion thereof.

13. The aquatic organism of claim 1, wherein the recombinant protein is acidic ribosomal phosphoprotein.

14. The aquatic organism of claim 1, wherein the enclosed aquatic system is completely isolated.

15. The aquatic organism of claim 1, wherein the enclosed aquatic system is partially isolated.

16. A method for producing an isolated recombinant protein in an aquatic organism comprising introducing DNA encoding an exogenous protein into the aquatic organism, culturing the aquatic organism in an enclosed aquatic system, harvesting the aquatic organism, and isolating the recombinant protein from the aquatic organism.

17. The method of claim 16, wherein the aquatic organism is a commercially cultured fish.

18. The method of claim 17, wherein the fish is selected from tilapia, sea bream, bass, zebrafish, rainbow trout, carp, cod, catfish, salmon, yellow tail, and red drum.

19. The method of claim 16, wherein the aquatic organism is a crustacean.

20. The method of claim 19, wherein the crustacean is an Artemia.

21. The method of claim 19, wherein the crustacean is a shrimp.

22. The method of claim 16, wherein the aquatic organism is a rotifer.

23. The method of claim 16, wherein the DNA comprises a promoter that is functional in the aquatic organism.

24. The method of claim 16, wherein the aquatic organism is transformed with a baculovirus vector.

25. The method of claim 24, wherein the baculovirus vector is Autographa californica nuclear polyhedrosis virus.

26. The method of either of claims 24-25, wherein the DNA comprises an early/intermediate promoter.

27. The method of either of claims 24-25, wherein the DNA comprises a late promoter.

28. The method of claim 16, wherein the recombinant protein comprises a post-translational modification.

29. The method of claim 16, wherein the DNA is introduced by infection.

30. The method of claim 16, wherein the DNA is introduced by microinjection.

31. The method of claim 30, wherein the DNA is introduced into an Artemia cyst by microinjection.

32. The method of claim 31, wherein the Artemia cyst is decapsulated, hatched, and cultured.

33. The method of claim 31, wherein the cyst is harvested by centrifugation.

34. The method of claim 16, wherein the aquatic organism is cultured in a completely isolated enclosed aquatic system.

35. The method of claim 16, wherein the aquatic organism is cultured in a partially isolated enclosed aquatic system.

36. The method of either of claims 34-35, wherein the aquatic organism is cultured in a recirculating aquatic system.

37. The method of claim 16, wherein the aquatic organism is harvested by filtration.

38. The method of claim 16, wherein the aquatic organism is harvested with nets.

39. The method of claim 16, wherein the aquatic organism is rapidly frozen upon harvest.

40. The method of claim 16, wherein the recombinant protein is thrombin.

41. The method of claim 40, wherein the recombinant protein is human thrombin.

42. The method of claim 16, wherein the recombinant protein is fibrinogen.

43. The method of claim 16, wherein the recombinant protein is green fluorescent protein.

44. The method of claim 16, wherein the recombinant protein is acidic ribosomal phosphoprotein or fusion thereof.

45. The method of claim 16, wherein the protein is targeted to a specific tissue, comprising inserting exogenous DNA under the control of a regulatory promoter endogenous to that tissue.

46. The method of claim 45, wherein the tissue is muscle.

47. The method of claim 45, wherein the tissue is skin.

48. A prophylactic and therapeutic method for treating a human or non-human animal, comprising feeding to the animal a crustacean as claimed in any of claims 1-15.

49. A prophylactic and therapeutic method for using the protein produced by the method of claim 16, comprising administering the protein to a human or non-human animal.
